(19) United States
(12) Patent Application Publication
(10) Pub. No.: US 2003/0064936 A1
(43) Pub. Date: Apr. 3, 2003

(54) Use of sphingoid base for inhibiting ceramidase activity

(76) Inventors: Willem F. Nieuwenhuizen, Bunnik (NL); Wim Van der Wilden, Rotterdam (NL); Maarten R. Egmond, Utrecht (NL); Inge Oudenaarde, Capelle a/d Ijssel (NL)

Correspondence Address: SCULLY SCOTT MURPHY & PRESSER, PC, 400 GARDEN CITY PLAZA, GARDEN CITY, NY, 11530

(21) Appl. No.: 10/232,466

(22) Filed: Aug. 30, 2002

(30) Foreign Application Priority Data
Aug. 31, 2001 (EP) 01203311.4

(51) Int. Cl. A61K 31/737; A61K 31/685

(52) U.S. Cl. 514/23; 514/78

(57) ABSTRACT

The present invention relates to the use of an active compound including a sphingoid base, a derivative of a sphingoid base, a salt of a sphingoid base, a ceramide or a mixture of two or more of these compounds for manufacturing a composition for topical application for inhibiting mammalian ceramidase activity in the skin and microbial ceramidase originating from micro-organisms present on the skin. The composition may further contain an amount of a compound capable of complexating divalent cations.

### DESCRIPTION

### FIELD OF THE INVENTION

The present invention relates to the use of a topical composition which includes an active compound that affects the activity of ceramidases that are responsible for unwanted hydrolysis of ceramides. The present invention also provides a method for inhibiting topically occurring ceramidase activity using the composition of the present invention.

### BACKGROUND OF THE INVENTION

Lipid lamellar structures in the intercellular spaces of the stratum corneum are reported to be responsible for the permeability function of the stratum corneum, in limiting loss of body fluids, in particular water, through mammalian skin and limiting penetration of foreign external compounds through the skin (see, for example, Elias, P. M., J. Invest. Derm. 1983, 80, 44-49). Ceramides are assumed to be the main lipid components within the intercellular lipid membranes and to be essential components in building and maintaining the barrier function of the stratum corneum. Other lipid components contributing to this barrier function include free fatty acids (mainly long chain free fatty acids), cholesterol, cholesteryl esters and cholesteryl sulfate.

The stratum corneum is built up of corneocytes that are received in a matrix of stacks of intercellular lipid membrane layers. Ceramides, as components of the lipid barrier, are thought to be responsible for holding the corneocytes together, similar to the role of mortar located between the bricks in a wall. Synthesis of ceramides takes place in the lower part of the stratum corneum. At the upper part of the stratum corneum, the ceramides are subjected to the action of ceramidases, which catalyze the hydrolysis of the ceramide N-acyl bond to generate a free fatty acid and a sphingoid base. In normal skin, equilibrium exists between ceramide synthesis and ceramide breakdown. Besides ceramidases, proteases which are also present on the surface of mammalian skin play a role in permitting the corneocyts to squame off in a controlled way so as to remove old cells from the skin. The term ‘squame off’ is used herein to describe the process of desquamation which represents a step in the regular process of skin removal, without referring to a disease. In the course of desquamation, old skin particles are released and replaced by new skin.

Various factors may adversely effect the ceramide content and thus the barrier function of the stratum corneum, such as, for example, exposure of skin to detergents or solvents, ageing of skin, or exposure to UV-light. A damaged barrier function will result in sensitive skin that, for example, looks red, dry and/or scaly. Moreover, a damaged barrier function to the stratum corneum may result in skin diseases, like (atopic) dermatitis, psoriasis, xerosis or ichthyiosis.

It has been observed that mammalian skin is typically populated with a wide variety of micro-organisms, including bacteria, yeasts and fungi. These micro-organisms secrete a wide variety of enzymes to the skin, including ceramidases. In this respect, Pseudomonas has been found to be responsible for the release of an alkaline ceramidase on the stratum corneum. Ceramidases originating from Pseudomonas have been isolated from patients with atopic dermatitis (see, for example, Nozomu Okino, J. Biol. Chem., 1999, Vol. 274, 51, pp 36616-36622).

EP-A-695,167 discloses a composition for topical application, which is thought to be capable of increasing the ceramide content in the stratum corneum by stimulating ceramide synthesis in the epidermis. The composition of EP-A-695,167 contains one or more ceramide synthesis pathway intermediates or precursors thereof. Topical application of these compounds is supposed to promote the ceramide synthesis rate-limiting step in which serine and palmytoyl-CoA are converted to 3-ketosphinganine. Disclosed ceramide pathway intermediates or precursors thereof include: N-acyl or O-acyl derivatives of sphinganine, sphingosine or phytosphingosine or mixtures thereof, for example, tetra-acetylphytosphingosine. The prior art composition may further comprise ceramide pathway adjuncts, for example, saturated or unsaturated α-, β-, or ω-hydroxy fatty acids, cholesterol, cholesterol pathway intermediates or precursors thereof, ceramides etc.

From J. Biolo. Chem, 1995, 270(21), 12677-84, the ability of the sphingosine reaction product for inhibiting ceramidase originating from guinea pig epidermis, and its catalytic activity in the hydrolysis reaction of ceramide to sphingosine is known. It is suggested that a regulatory mechanism exists for balancing the ratio of ceramide/sphingosine which can serve as an intracellular effect in the epidermis. In Biochemistry 14/8/2001, 40(32): 9657-68, it is disclosed that sphingosine is capable of inhibiting ceramidase originating from mitochondria of rat brain. To obtain inhibition, the enzyme requires the primary and secondary hydroxyl group, the C₄-C₅ double bond, the trans configuration and the protons of the NH₂ group. The aforementioned publications relate to a sort of in-vitro experiments. According to JP10014563, the bacterium causing atopic dermatitis produces ceramidase and compounds capable of de-activating ceramidase should be capable of treating atopic dermatitis. This assumption is deducted from experiments in which ceramidase was decomposed by subjecting it to heat-treatment. According to Clin. Diagn. Lab. Immunol. 1999, 6(1), 101-104, patients suffering from atopic dermatitis secrete more ceramidase from the skin than healthy people. None of these publications describe any enzyme inhibiting activity of sphingoid bases, by topical application thereof.

WO94/23694 discloses that ceramide synthesis in the epidermis may be stimulated and the ceramide content of the epidermis may be increased through topical application of ceramide precursors or pathway intermediates.

### SUMMARY OF THE INVENTION

The applicants have now analyzed phenomena underlying the problem of an impaired permeability barrier function of skin. It appeared to the applicants that an impaired barrier function may be associated with a decreased ceramide content of the skin following increased ceramidase catalyzed hydrolysis of the ceramide N-acyl bond. This ceramidase activity is thought to be responsible for distorting the equilibrium between ceramide synthesis and ceramide decomposition. There is thus a need to provide a composition, which is capable of inhibiting the activity of the ceramidases responsible for the unwanted hydrolysis of ceramides.

Accordingly, it is an object of the present invention to provide a composition which is capable of affecting the activity of ceramidases responsible for the unwanted hydrolysis of ceramides. This is achieved in the present invention by topical application of a composition with the technical features described herein below. According to the present invention, an active compound comprising sphingoid bases, a derivative of a sphingoid base, a salt of a sphingoid base, a ceramide or a mixture of two or more of these compounds is used for manufacturing a composition for topical application for inhibiting ceramidase activity in and/or on mammalian skin.

It has been surprisingly found that topical application of the composition of the present invention has the effect that the activity of mammalian ceramidases present in the skin as well as the activity of microbial ceramides secreted by micro-organisms present on the skin, may be inhibited. The fact that the active compound of the present invention is capable of inhibiting both mammalian and microbial ceramidase is surprising since it had been observed by the applicants that ceramidases of microbial origin do not respond to inhibition by well-known mammalian ceramidase inhibiting compounds. Examples of such mammalian ceramidase inhibiting compounds, include, but are not limited to: D-erythro-MAPP ((1S,2R)-D-erythro-2-(N-myristoylamino)-1-phenyl-1-propanol) disclosed in U.S. Pat. No. 5,830,916, or N-oleolyethanolamine (see, for example, Sugita et al. Biochim Biophys Acta 398: 125 (1975)). The present finding that the composition of the present invention is capable of inhibiting mammalian as well as microbial ceramidase may point to a still unknown similar active site and/or substrate-binding region present in mammalian and microbial ceramidase.

As there is a continuous synthesis of ceramides in the epidermis, inhibiting the ceramide decomposition will have the effect that a lacking amount of ceramide will be replenished and the over-all ceramide content of the stratum corneum may be maintained, restored and even increased. The present invention in fact provides a novel approach to repairing a damaged skin barrier. According to the present invention, the problem of the damaged barrier function is not solved by replenishing the lacking compound through topical application, but rather by treating and inhibiting the underlying effect causing damage of the skin barrier, namely the ceramidase catalyzed decomposition of ceramides. Because the composition of the present invention is capable of penetrating skin, activity by microbial ceramidase secreted on top of the skin as well as ceramidase present in the stratum corneum may be inhibited.

Increasing the ceramide in the stratum corneum content has the effect that a locally distorted lipid barrier may be repaired and the lipid barrier may even be improved, which, in the end, will result in a thickening of the skin and an improvement of the over-all condition and quality of the skin. The increased ceramide content in the stratum corneum further allows preventing or repairing the occurrence of dry, itchy and/or rough skin, as well as reducing the occurrence of problems with skin sensitivity or skin diseases. Due to this thickening, ageing of the skin as well as the phenomena associated therewith, such as the occurrence of fine lines or wrinkles, may be retarded. Also, inhibiting ceramide decomposition will assist in counteracting the occurrence of, for example, a dry or solvent affected skin, following impaired barrier function as a result of a decreased ceramide content.

It is known that environmental factors, like for example, chemicals, detergents and UV rays may have a detrimental effect on the functioning of the skin barrier. Because skin ceramidases act on the boundary between skin and the environment, these environmental factors may contribute to, and even enhance, the effect of ceramidases, thus increasing ceramide decomposition and deteriorating the barrier function of the skin. Further, the external factors, as well as the consequential damaged skin barrier, may involve a local increase in the number of micro-organisms on the skin. As a consequence, the amount of microbial ceramidase secreted on that part of the skin will be increased. It is likely that following an overcolonization of micro-organisms on parts of the skin, the ceramidase concentration is locally increased and involves a locally distorted balance between ceramide synthesis and breakdown. The increased ceramide hydrolysis may lead to a ceramide level in the stratum corneum, which is insufficient to maintain the barrier function at the required level.

It is known from EP-A-814,075 that sphingoid bases are able to inhibit topical growth of micro-organisms on the skin. As a consequence, topical application of the composition of the present invention will not only have the effect that ceramidase activity is inhibited, but also that the growth of micro-organisms is counteracted. The decreased population by micro-organisms, in turn, will have the effect that the amount of ceramidase of microbial origin secreted on the skin will be minimized. Topical application of the composition of the present invention thus produces a dual effect in that not only the concentration of the barrier lipids in the stratum corneum may be increased by inhibiting their decomposition by ceramidases, but also that the overcolonization of microbial flora will be counteracted by inhibiting the growth of the micro-organisms, thereby decreasing the amount of secreted ceramidases.

Benefits perceived by the consumer may be seen from improved skin barrier function involving fewer problems of skin sensitivity, skin diseases (such as atopic or diaper dermatitis, psoriasis, or ichthyiosis), photodamaged skin, loss of elasticity and flexibility, together with an improved skin condition, removal of age spots, keratoses, wrinkles, fine lines, coarse, rough and dry skin.

The observation that sphingoid bases, salts thereof or derivatives thereof are capable of inhibiting ceramidase activity has initiated a novel method of selectively treating increased ceramidase activity of mammalian and microbial origin in and on the skin, thereby maintaining, repairing or even enhancing the barrier function of the skin.

Mao C. et al (J. Biol. Chem., 2001; 276 (28): 26577-88) reported that expulsion of a human alkaline ceramidase (aPHC) in a yeast mutant strain caused an increased hydrolysis of phytoceramide in yeast cells. The human alkaline ceramidase has phytoceramidase activity both in vitro and in cells. Although it has been disclosed that human alkaline ceramidase is inhibited by sphingosine in vitro and in cells, it has not been reported or taught in the prior art that topical application of sphingosine results in an inhibition of both mammalian and microbial ceramidase. Additionally, the prior art has not disclosed or taught that the ceramide content of the stratum corneum and the barrier function of the skin may be maintained, repaired or improved as a result of ceramidase-inhibition.

### DETAILED DESCRIPTION OF THE INVENTION

The present invention provides a composition that inhibits mammalian ceramidase activity in the skin as well as inhibiting ceramidase originating from micro-organisms present on the skin. The composition of the present invention, which is applied topically to mammalian skin includes, as an active compound, a compound selected from the group consisting of a sphingoid base, a salt of a sphingoid base, a ceramide and a mixture of two or more of these compounds.

This inhibitory effect has been found to be caused by one of the above-mentioned active compounds, preferably from sphingoid bases or derivatives or salts thereof or a mixture of two or more of these compounds. The sphingoid bases are preferably selected from phytosphingosine, sphinganine, sphingosine, and 6-hydroxy-sphingosine. The active compound is preferably a D-enantiomer since D-enantiomers are the stereochemical configuration of the compound found in the mammalian body.

The composition of the present invention preferably has a pH within the range of 4-9. The following groups of ranges are more preferred in the present invention: pH between 5-7; pH between 4 and 5; pH between 7 and 9; or pH between 8 and 9, since these are the pH-ranges in which various types of ceramidases may be active.

The composition of the present invention also preferably includes a compound which is capable of complexating divalent cations. The capturing of these divalent cations, which are essential co-factors for the function of ceramidase, prevents ceramidases from properly functioning. These divalent cations may originate from various sources and may, for example, be present on the skin, or be deposited on the skin from external sources. The simultaneous application of the active compound of the present invention and the complexating agent has been found to produce a synergistic effect, according to which an improved inhibition of microbial ceramidase activity may be achieved.

The nature of the cation complexating compound is not critical to the present invention and may be selected from the complexating agents generally known to those skilled in the art. Because the composition of the present invention is intended for topical application, the complexating compound will preferably be selected such that it is a cosmetically or pharmaceutically acceptable ingredient, for example, EDTA (ethylenediaminetetraacetic acid) or citric acid. Other complexating compounds include di- and tri-carboxylic organic acids and fatty acids.

The composition of the present invention preferably comprises a protease inhibitor since such an inhibitor has a beneficial effect on the desquamation process of the skin. Since proteases interfere in controlling the squaming off of corneocytes from the skin, proteases also play an important role in maintaining the barrier function of the skin. The protease inhibitor will, in general, be present in a concentration of between 0.001-10 wt. %, preferably 0.01-5 wt. %.

The composition of the present invention may further include one or more of the following: a cosmetically acceptable vehicle; any additional compound capable of supplementing the effect of the active compound as well as the usual adjuvants.

The present invention further relates to the use of the above-described composition for manufacturing of a cosmetic agent or a medicament for inhibiting ceramidases of mammalian and/or microbial origin.

Also pets and/or farm animals may benefit from topical application of the inventive composition as such animals also suffer from itchy, dry, and/or scaly skin and/or skin diseases such as dermatitis.

The Active Compound

The active compound contained in the composition of the present invention, preferably includes sphingoid bases, a derivative of a sphingoid base, a salt of a sphingoid base, or a mixture of two or more of these compounds.

Examples of suitable sphingoid bases include phytosphingosine, sphinganine, sphingosine and 6-hydroxysphingosine. The free sphingoid base present in the composition according to the present invention has a general structure according to Formula 1:

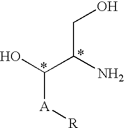

in which:

A is CH₂—CH₂—(R), CH═CH—(R), C(H)OH—CH₂—(R), or —CH═CH—CHOH—(R); and R is a straight chain or branched alkyl group having 10 to 22 carbon atoms which may optionally contain one or more double bonds and/or may optionally be substituted with one or more hydroxyl groups. More preferably, R is a straight chain alkyl group having 12 to 18 carbon atoms, with a straight chain alkyl group having 13 carbon atoms being most highly preferred. Both asymmetric carbon atoms, represented by the two * groups in Formula (1), may either take the D or L configuration.

The sphingoid bases used in the composition of the present invention may be obtained from any suitable source, e.g., from a natural source or from a chemical synthesis process. It is desirable to apply a production process in which the sphingoid base may be obtained in the correct stereochemical configuration, and in sufficient amounts at a commercially feasible price. Purification of animal and/or plant tissue extracts usually yields small amounts of sphingoid bases, making their isolation costly. Moreover, animal sources are believed to be unsafe due to the presence of viruses and other infectious agents, such as the agent causing BSE (mad cow's disease). Therefore, preferable use is made of sphingoid bases obtained from a microbial fermentation process, using Pichia ciferrii as a yeast (see, for example, the process disclosed in U.S. Pat. No. 6,204,006). Phytosphingosine may, for example, be obtained by a chemical or enzymatic catalyzed deacetylation of Pichia ciferrii-derived tetraacetyl-phytosphingosine (TAPS). Phytosphingosine resulting from alkaline TAPS hydrolysis may be purified by any method known to one skilled in the art. Yeast-derived phytosphingosine is human skin-identical, since it is reported to have the same stereochemical configuration as mammalian phytosphingosine, i.e., the D-erythro configuration.

Examples of salts of sphingoid bases suitable for use in the present invention include those disclosed in WO 00/53568. The entire content of WO 00/53568 is incorporated herein by reference. The anion of the sphingoid base salt is preferably derived from an acid, which when mixed with the sphingoid base and a suitable solvent, produces a salt with an improved solubility in an aqueous medium, and has an efficacy in topical application. The acid may be a hydrophilic organic acid, such as, an α-hydroxy alkanoic acid, a β-hydroxy alkanoic acid, an α,β-hydroxy alkanoic acid, an alkanedioic acid or a mineral acid. Suitable organic acids include, but are not limited to: lactic acid, glycolic acid, malic acid, pyruvic acid, succinic acid, fumaric acid, citric acid, ascorbic acid, gluconic acid and/or pyroglutamic acid (pyrrolidone carboxylic acid), and lipophilic organic acids. Preferred examples of mineral acids include, but are not limited to: hydrochloric acid, nitric acid and/or phosphoric acid.

Examples of derivatives of sphingoid bases include compounds corresponding to the formula:

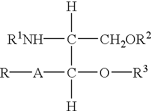

in which

A is CH₂—CH₂—(R), CH═CH—(R), C(H)OR⁴—CH₂—(R), or —CH═CH—CHOR⁴—(R) where R is as defined above;

R¹, R², R³, R⁴ are each individually represented by H—, a straight or a branched alkyl chain having between 1 and 30 carbon atoms, CH₃(CH₂)ₙ(CHOH)ₘ—, CH₃(CH₂)ₙ(CHOH)ₘC(═O)—, CH₃(CH₂)ₙC(═O)—, a B—O—D—C(═O) residue, in which D is a C₁₅₋₃₅ straight chain alkyl group which may optionally contain one or two double bonds, and B is a C₁₂₋₂₀ straight chain acyl group which may optionally contain one or two double bonds, for example, lineoyl, stearoyl, oleoyl, phosphorylated, sulfated, glycosylated, benzoyl, —CO—C₅H₄OH (salicylic acid), retinoic acid, derivatives thereof, a 2-α-hydroxy acid residue having 3-40 carbon atoms, for example, lactic acid, glycolic acid, methyllactic acid, alpha hydroxycaprylic acid, alpha-hydroxylauric acid, or derivatives of these compounds; n is 0 or an integer between 1-30, preferably between 1-22; and m is 0 or an integer between 1 and 5.

Examples of preferred sphingoid base derivatives for use in the present invention include: retinoylamide derivatives of sphingoid bases disclosed in EP-B-789,686; and the lactic acid, and salicylic acid derivatives disclosed in EP-A-966,431. The entire contents of the '686 and '431 references are incorporated herein by reference. The more lipophilic character of the retinoylamide derivatives enhances penetration into skin as compared to an unmodified retinoid.

Other preferred derivatives of sphingoid bases include their respective N-acyl, O-acyl and N-alkyl derivatives, containing a C₁-C₂₄ acyl or alkyl group. Particularly preferred sphingoid base derivatives include, for example: tetraacetyl-, triacetyl-, diacetyl phytosphingosine (TAPS), N-monoacetyl phytosphingosine (MAPS), triacety-, diacetyl and N-monoacetyl sphingosine, triacetyl-diacetyl and N-monoacetyl sphinganine. In some embodiments, the N-atom may be dialkylated.

Suitable ceramides for use as an active component in the present invention are preferably selected from the group consisting of ceramide 1, 1A, 2, 3, 3A, 3B, 4, 5, 6, 7 and 8. The ceramides 1, 1A, 2, 3, 3A, 3B, 4, 5, 6, 7 and 8 refer to specific ceramides which correspond to the more general formulas which appear herein above in which R¹ is CH₃(CH₂)ₙ(CHOH)ₘCO or CH₃(CH₂)ₙCO. It is noted that the terminology ceramide 1, 1A, 2, 3, 3A, 3B, 4, 5, 6, 7, or 8 is an art recognized way of describing ceramides. See, for example, S. A. Long, et al., Human Stratum Corneum Polar Lipids and Desquamation”, Archl. Dermatol: Res. (1985) 277:284:287. More specifically, the ceramides used in the present invention comprise a sphingoid base backbone selected from the group consisting of sphingosine, 6-hydroxy sphingosine, phytosphingosine and sphinganine. The sphingoid base backbone may be acylated with an acyl or an acyloxyacyl group, which may have a variable chain length, be branched or not, contain one or more double bonds, or contain one or more hydroxyl groups. Preferred ceramides may be selected from the group consisting of N-tetracosanoyl phytosphingosine, N-stearoyl phytosphingosine, N-oleoyl phytosphingosine, N-linoleoyl-phytosphingosine, N-(2-hydroxytetracosanoyl) phytosphingosine, N-(2-hydroxyoctadecanoyl) phytosphingosine, N-(27-stearoyloxyheptacosanoyl) phytosphingosine, N-(27-oleoyloxyheptacosanoyl) phytosphingosine, N-(27-linoleoyloxyheptacosanoyl) phytosphingosine, and N-(23-stearoyloxytricosanoyl) phytosphingosine.

The active component used in the present invention may also be made of a sphingolipid, for example, a glycoceramide. With respect to glycoceramides, two groups of these compounds are typically distinguished, i.e., cerebrosides and gangliosides. A cerebroside is understood to be a glycoceramide wherein a monosaccharide, mostly glucose or galactose, is attached to the oxygen of the —CH₂OH group. In gangliosides, oligosaccharides frequently including sialic acid, are attached to the same.

When incorporated into a formulation, the concentration of the active compound of the present invention will preferably be varied from 0.0001-15 wt. % to allow achieving the desired inhibitory effect. More preferred ranges include: between 0.001-10 wt %, 0.01-5 wt. % and 0.01-2.5 wt. % since these doses are capable of achieving the required inhibitory effect, while minimizing the cost of the composition.

The Cosmetically Acceptable Vehicle

The composition of the present invention also comprises an amount of a cosmetically acceptable vehicle, which may be in a solid, semi-solid, viscous, or liquid form to enable topical application of the composition. A person skilled in the art will be capable of selecting the appropriate vehicle depending on the intended application and way of administration.

Vehicles other than water can include liquid or solid emollients, solvents, humectants, thickeners, powders, surfactants, which are also sometimes designated as emulsifiers, solubilizers, propellants and other active ingredients.

Suitable emollients include, for example, (fatty acid) esters, fatty acids, (fatty) alcohols, polyols, (natural) waxes, natural oils, silicone oils including volatile and non-volatile silicone oils, and hydrocarbons such as mineral oil, petroleum jelly, vaseline, squalens and (iso)paraffin.

Additional Ingredients

The nature of the complexating compound for capturing divalent cations is not critical to the present invention and thus may be selected by one skilled in the art from a wide variety of known complexating agents. Suitable examples include: EDTA, citric acid, or any cosmetically or pharmaceutically acceptable organic acid containing two or three or even more carboxylic acid groups.

The cation complexating compound is preferably present in an amount between 0.0001-25 wt. % to allow for achieving the desired inhibitory effect. However, depending on the molecular weight of the complexating compound, the concentration may also be varied from 0.005-5% or from 0.01-1 wt. %.

The composition of the present invention optionally may comprise one or more additional skin lipid compounds, such as, cholesterol, cholesterol esters like cholesteryl sulfate, free fatty acids like palmitic, stearic, behenic, oleic and/or linoleic acid and/or other sphingolipids.

Other additional ingredients that can be present in the composition of the present invention include those compounds, which as such, have beneficial effect to the skin. For instance, yeast β-glucan may be applied in the composition to reduce UV-induced erythema.

Suitable propellants for use in the present invention include, for example, propane, butane, isobutane, dimethyl ether, chlorofluoroalkanes, carbon dioxide, and nitrous oxide.

Solvents which may be used in the present inventive include, but are not limited to: ethyl alcohol, methylene chloride, isopropanol, ethyl ethers such as ethoxyethanol and butoxyethanol, acetone, tetrahydrofuran, dimethylformamide, DMSO (dimethyl sulfoxide), propylene glycol, and butylene glycol.

The present invention contemplates that the composition may also include various humectants, such as, proteins and protein hydrolyzates, amino acids, sorbitol, glycerin, sorbitol, glycols preferably PEG (polyethylene glycol) 200-4000 and other polyols.

Another component that may be employed in the present invention is a thickener, such as, cross linked polyacrylates, silicone gums and polysaccharide gums such as xanthan, carrageenan, gelatin, pectin and locust beans gum, hyaluronic acid and carboxylic group-containing polymers.

Powders including chalk, talc, starch, kaolin, clays, silicates, or carboxyvinyl polymers may also be employed in the inventive composition.

Other active ingredients that are also contemplated to be used in the present invention include:

anti-oxidants like butyl hydroxy toluene, hydroquinone, tocopherols, and gallates;

preservatives like para-hydroxy benzoate esters, sorbic acid, quaterniums, benzoic acid, imidazolidinyl urea, and (benzyl)alcohol;

sunscreens, blocking UV light, like PABA's (para-aminobenzoic acids), and cinnamate.

The combination of the above-mentioned components can account for 5 to 99% of the composition.

Use of the Composition

The composition of the present invention is intended for topical application to skin. The term ‘skin’ is used herein to denote human as well as animal skin, including the human scalp and animal fur. Topical application is understood to denote cosmetic and/or dermatological application on skin, on hair and on the epithelial linings of the mouth, nose, eye, urogenital tract, and the like.

The composition may be used as such, or be incorporated in a further formulation for topical application. The composition of the present invention is intended for use as a cosmetic agent for preventing, reducing, or treating the signs of ageing of the skin, thin skin, fine lines and/or wrinkles, sensitive skin, induced coarse, dry and/or scaly, damaged skin, repairing skin barrier function where this is imparted by increased ceramidase activity and, in general, for improving quality of skin. The composition of the present invention is also intended for use as a pharmaceutical composition for treating skin diseases such as, for example, dermatitis, psoriasis, and ichtyosis.

If microbial ceramidase activity is negligible, the composition of the present invention may be used for exclusively controlling mammalian ceramidase activity.

Topical compositions according to the present invention comprising the above-mentioned active ingredient may be applied in the form of a cream, lotion, gel, emulsion, paste, ointment, aerosol, spray, spot-on, shampoo, plaster, dressing, and other forms of application known to one skilled in the art.

Examples of topically occurring undesirable and/or abnormal conditions in which topical compositions comprising the active ingredient of the present invention are advantageously applied include: psoriasis, acne, dandruff, mouth and/or lip infections, mycoses, various other skin-infectious diseases or vaginal infections, wound-healing, normalization of skin flora.

Through topical application of the composition of the present invention a synergistic effect may be achieved. According to this effect, a maintaining, restoring, repairing or even an improvement of the ceramide content of the skin may be achieved, by treating the phenomenon causing ceramide decomposition. On one hand, topical application of the composition of the present invention allows for inhibiting the activity of ceramidases naturally present in the skin, as well as the activity of ceramidases secreted by micro-organisms present on the skin. Besides this, topical application of the composition of the present invention also allows for controlling the amount of micro-organisms present on the skin by inhibiting their growth. In that way, the amount of microbial ceramidase released to the skin may be controlled. All of the above mentioned effects contribute to curing repairing, enhancing or maintaining the barrier function of the skin.

The present invention also relates to a method for inhibiting topically occurring ceramidase activity, comprising the topical application of the topical composition mentioned above.

The present invention is further described in the following examples and comparative examples.

### EXAMPLES

The effect of various active compounds on the enzymatic activity of recombinant ceramidase from Pseudomonas aeruginosa PA01 was tested.

The assays were performed at 25° C., with magnetic stirring, using Photo Technology International fluorimeter equipped with a LPS 220 Lamp Power Supply, a 810 Photomultiplier Detector System, a MD 5020 Motor Driver, and a Dell Dimensions M166a computer with FELIX® software. Excitation was done at 346 nm (±2 nm) and the emission was measured at 378 nm (±2 nm).The assay buffer consisted of 50 mM Tris/HCl, 4 mM CaCl₂, 0.1% w/v Triton X-100, pH 8.5.

The active compounds were dissolved in DMF (dimethylformamide) to a concentration of 4 mM. Of these solutions, 250 μliter was added to 50 milliliter assay buffer under vortex, yielding 20 μM lipid solutions.

To 1 mL of the lipid solution in a cuvet, 5 μL of quenched fluorescent ceramide analog as a substrate was added (end concentration 2 μM) and the system was allowed to equilibrate for 5 minutes. Then 5 μL of recombinant ceramidase was added (end concentration 4.3 nM). The reaction was allowed to proceed for 20 minutes. The slope at 378 nm was used to determine the reaction rate.

Control experiments were done in the absence of sphingolipids, with corresponding amounts of DMF (0.5% v/v for the 20 μM samples). The reaction rate of the controls was set at 100%. All assays were done in duplo, and the average values were used to calculate the values given in the table below. The percentages in the table are given as % of inhibition (control value—reaction rate of the sample).

### Comparative Example I

The potential of several known human ceramidase inhibiting compounds of inhibiting Pseudomonas aeruginosa PA01 ceramidase was tested at various concentrations, in the assay buffer containing 20 μM of substrate.

The microbial inhibiting potential of the following compounds was determined: D-erytro-MAPP, L-erythro-MAPP (the inactive enantiomer of D-erytro-MAPP), N-oleoylethanolamine, L(−)-norephenidrin, D(+)-norephenidrin. None of these compounds was found capable of inhibiting the Pseudomonas aeruginosa ceramidase.

### Example 1-9

The potential of several ceramide and sphingoid bases of inhibiting Pseudomonas aeruginosa PA01 ceramidase was tested at a pH of 8.5, using respectively, 2 μM and 20 μM of ceramidase substrate, 1.6 mM Triton X-100 and 4 mM of CaCl₂. The ability of the active compounds of inhibiting ceramidase activity are given in % of inhibition (control value—reaction rate of the sample).

As can be seen from the table, D-erythro sphingosine (example 8) shows the highest ceramidase inhibition ability, followed by the mixture of D and L sphinganine (example 7). Based on the observation that the inhibiting effect produced by L-erythro-sphingosine is lower as compared to the D-enantiomer, it is expected that the inhibiting effect of D-sphinganine will be better than the inhibiting effect of the racemic mixture.

Phytosphingosine.HCl was found to have a better ceramidase inhibiting effect than phytosphingosine (example 2 and 3).

From examples 8 and 9, it appears that the ceramidase has a stronger chiral preference for the binding of D-erythro sphingosine as compared to the L-enantiomer, as is reflected in the different inhibiting power of both enantiomers.

| | |
|---|---|
| Concentration of | % of inhibition |

| active compound | 20 μM | 2 μM |
|---|---|---|
| Active compound | | |
| 1. Ceramide VI | 11 | |
| 2. Phytosphingosine | 60 | 7.5 |
| 3. Phytosphingosine.HCl | 67 | 15 |
| 4. N—acetyl—phytosphingosine | 9 | |
| 5. MAPS | 35 | |
| 6. TAPS | 22 | |
| 7. D/L sphinganine | 83 | |
| 8. D—erythro—sphingosine | 97 | |
| 9. L—erythro—sphingosine | 20 | |

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A method for treating skin comprising applying a topical composition comprising at least an active compound selected from the group consisting of a sphingoid base, a derivative of a sphingoid base, a salt of a sphingoid base, a ceramide and a mixture of two or more of said compounds to said skin, wherein said active compound is used for inhibiting mammalian ceramidase and microbial ceramidase originating from micro-organisms.

2. The method of claim 1 wherein said active compound is a sphingoid base selected from the group consisting of sphingosine, 6-hydroxysphingosine, phytosphingosine, sphinganine and a mixture of two or more of these compounds.

3. The method of claim 1 wherein said active compound is a sphingoid base derivative selected from the group consisting of a N-salicyloyl-sphingoid base, N-lactyloyl-sphingoid base, N-retinoyl-sphingoid base, N-acetyl sphingid base, diacetyl-, triacetyl- or tetraacetyl sphingoid base, di- or triacectyl phytosphingosine, di- or triacetyl sphinganine and a mixture of two or more of these compounds.

4. The method of claim 1 wherein said active compound is a sphingoid base salt selected from the group consisting of sphinhoid base salts of ascorbic acid, citric acid, fumaric acid, gluconic acid, glycolic acid, hydrochloric acid, lactic acid, malic acid, pyroglutamic acid and a mixture of two or more of these compounds.

5. The method of claim 1 wherein said active compound is a ceramide selected from the group consisting of ceramide 1, 1A, 2, 3, 3A, 3B, 4, 5, 6, 7, 8 and glycoceramides.

6. The method of claim 1 wherein said active compound has a D-erythro stereochemical configuration.

7. The method of claim 1 wherein said active compound is used in conjunction with a compound capable of complexating divalent cations.

8. The method of claim 7 wherein said complexating compound is selected from the group consisting of di- and tri-carboxylic organic acids, fatty acids, EDTA, and citric acid.

9. The method of claim 1 wherein said composition has a pH of between 4 and 9.

10. The method of claim 1 wherein said composition has a pH of between 5 and 7.

11. The method of claim 1 wherein said active compound is present in a concentration of between 0.0001-15 wt. %

12. The method of claim 1 wherein said active compound is present in a concentration of between 0.001-10 wt. %.

13. The method of claim 1 wherein said active compound is present in a concentration of between 0.01-2.5 wt. %.

14. The method of claim 1 further comprising adding a protease inhibiting agent to said composition.

15. The method of claim 1 wherein said active compound maintains, repairs, restores or enhances a permeability barrier function of the skin which has been disturbed by ceramidase activity.

16. The method of claim 1 wherein said active compound prevents, reduces or treats signs of ageing of the skin, thin skin, fine lines, wrinkles, sensitive skin, dry skin or scaly skin.

17. The method of claim 1 wherein said active compound inhibits ceramidase activity in and on mammalian skin, scalp, or a combination thereof.

18. The method of claim 1 wherein said active compound inhibits ceramidase activity in and on animal skin and fur.

19. The method of claim 1 wherein said composition is formulated into a cosmetic composition.

20. The method of claim 1 wherein said composition is formulated into a medicament.

21. A method for inhibiting topically occurring ceramidase activity, comprising topically applying a composition comprising an active compound selected from the group consisting of a sphingoid base, a derivative of a sphingoid base, a salt of a sphingoid base, a ceramide, and a mixture of two or more of these compounds to skin or scalp.

22. The method of claim 21 wherein said active compound is a sphingoid base selected from the group consisting of sphingosine, 6-hydroxysphingosine, phytosphingosine, sphinganine and a mixture of two or more of these compounds.

23. The method of claim 21 wherein said active compound is a sphingoid base salt selected from the group consisting of sphinhoid base salts of ascorbic acid, citric acid, fumaric acid, gluconic acid, glycolic acid, hydrochloric acid, lactic acid, malic acid, pyroglutamic acid and a mixture of two or more of these compounds.

24. The method of claim 21 wherein said active compound is a ceramide selected from the group consisting of ceramide 1, 1A, 2, 3, 3A, 3B, 4, 5, 6, 7, 8 and glycoceramides.
